(19) United States
(12) Patent Application Publication
(10) Pub. No.: US 2005/0033189 A1
(43) Pub. Date: Feb. 10, 2005

(54) Electrophysiological intuition indicator

(76) Inventors: Rollin I. McCraty, Boulder Creek, CA (US); Michael A. Atkinson, Boulder Creek, CA (US); Doc L. Childre, Boulder Creek, CA (US)

Correspondence Address: CROWELL & MORING LLP;INTELLECTUAL PROPERTY GROUP, P.O. BOX 14300, WASHINGTON, DC, 20044-4300, US

(21) Appl. No.: 10/912,838

(22) Filed: Aug. 6, 2004

Related U.S. Application Data
(60) Provisional application No. 60/493,936, filed on Aug. 8, 2003.

(51) Int. Cl. A61B 5/04

(52) U.S. Cl. 600/509

(57) ABSTRACT

Systems and methods for electrophysiological detection and measurement of intuition are disclosed. In one embodiment, one or more electrophysiological properties of one or more individuals are monitored and used as an indication of a future event. In one embodiment, the electrophysiological property may include heart rate variability, brain wave activity, respiration pattern, skin conductance level, etc. In another embodiment, a signal averaging technique is used to generate a waveform that may be used as an indicator of future events.

### CROSS REFERENCE TO RELATED APPLICATIONS

This application is related to and claims the benefit of U.S. provisional patent application No. 60/493,936, filed on Aug. 8, 2003. This application also claims priority to U.S. patent application Ser. No. 10/486,775 which is based upon PCT International Application No. PCT/US00/05224, filed on Mar. 1, 2000, which claims the benefit of U.S. patent application Ser. No. 09/260,643, filed on Mar. 2, 1999, which is hereby fully incorporated by reference.

### BACKGROUND OF THE INVENTION

1. Field of the Invention

The present invention relates to detecting indications of intuition and more particularly, to systems and methods for electrophysiological detection and measurement of intuition.

2. Background of the Invention

It is commonly assumed among neuroscientists that mental concepts, conscious awareness, memory, and unconscious perception are emergent properties of the brain and nervous system. It is assumed that the mind is essentially a complex, dynamical system that is subject to standard physical constraints. Thus, the mind is assumed to be restricted to perceptions of present sensory input, intermingled with memories of the past. Intuition is thus often assumed to be related to information stored in the subconscious mind which can affect ones feeling or decisions at an unconscious level.

Within physics, however, an absolute direction of time is far less certain (e.g., general relativity, electrodynamics and quantum mechanics). These non-local effects are generally assumed to manifest only in subatomic realms. However, macroscopic scale examples have been reported throughout history (e.g., prophesy, precognition, gut instinct, intuition, etc.). For nearly a century, researchers have investigated these phenomena to determine if they are best understood as coincidence, selective memory, or what they appear to be—perception of non-inferable future events.

Of particular interest is the intuitive hunch, commonly described as a “bad feeling” with no evident cause, occurring before an unexpected emotional event. We have shown through rigorous methods that sometimes if a future event is sufficiently important, novel, or emotional, it may precipitate a change in the present physiological state that is consistent with the future reaction. One important aspect of this research has shown that there is a relationship between the emotionality of the actual event and the change in physiological status that can occur prior to the actual event. Thus electrophysiological measures of nervous system dynamics that reflect changes in emotional state are important aspects of detecting and measuring intuition.

We have also found that the clear rhythmic patterns in beat-to-beat heart rate variability (HRV) are distinctly altered when different emotions are experienced. In addition, there are specific changes that occur in short time scales (3 to 10 seconds) and longer time scales (10 seconds to minutes).

Heart rate variability (HRV), derived from the electrocardiogram (ECG), is a measure of the naturally occurring beat-to-beat changes in heart rate. The analysis of HRV, or heart rhythms, provides a powerful, noninvasive measure of neurocardiac function that reflects heart-brain interactions and autonomic nervous system dynamics, which are particularly sensitive to changes in emotional states

However, heretofore there has been no appreciation for the relationship between intuition detection and certain electrophysiological indicators, including HRV, EEG and ECG. Thus, there is a need in the art for an electrophysiological intuition indicator.

### BRIEF SUMMARY OF THE INVENTION

The present invention relates to systems and methods for electrophysiological detection and measurement of intuition. In one embodiment, the method comprises measuring the electrophysiological properties of a subject at a first point in time, and measuring the electrophysiological properties of said subject at a second point in time. The method further comprises calculating a measure of change of the electrophysiological property between the first point in time and the second point in time, and determining an event to occur at a third point in time based on the calculated measure. In one embodiment, determining an event involves predicting the probability of an event to occur at the third point in time based on the calculated measure.

Other embodiments are disclosed and claimed herein.

### BRIEF DESCRIPTION OF THE DRAWINGS

FIG. 1 is a graph illustrating how emotions are reflected in heart rhythm patterns;

FIG. 2 depicts a typical HRV power spectrum;

FIG. 3 depicts one embodiment of a procedure for implementing an intuition indicator;

FIG. 4 depicts a graph of electrophysiological data based on the procedure of FIG. 3;

FIG. 5 depicts yet another graph of electrophysiological data based on the procedure of FIG. 3;

FIGS. 6A-6B depict two embodiments of operational modes consistent with the principles of the invention;

FIG. 7A is a flow diagram for one embodiment of a calibration phase of the invention; and

FIG. 7B is a flow diagram for one embodiment of an application phase of the invention.

### DETAILED DESCRIPTION OF EXEMPLARY EMBODIMENTS

System and methods for electrophysiological detection and measurement of electrophysiological intuition indicators are disclosed. In one embodiment, one or more electrophysiological properties of an individual are monitored and used as an indication of an unknown or future event. In one embodiment, the electrophysiological property is the individual's HRV (heart rate decelerations and accelerations), while in other embodiments it may be the individual's brain wave activity as measured by an electroencephalogram (EEG), respiration pattern, skin conductance level (SCL), etc. One aspect of the invention is to utilize one or more electrophysiological properties of a group of individuals as a predictive tool for certain future events, such as investment decisions, gambling, etc.

In one embodiment, a “signal averaging” technique is a digital technique for separating a repetitive signal from noise without introducing appreciable signal distortion is used to detect EEG activity that is time-locked to ECG activity. In another embodiment, the resultant waveform is used to quantify the level of synchronization of brain activity to cardiac activity. Signal averaging techniques may be applied to the electrophysiological properties of one or more individuals. The resulting waveforms may then be used as indicators of the probability of future or unknown events actually occurring.

I. Terminology Overview

Heart rate variability (HRV), derived from the ECG, is a measure of the naturally occurring beat-to-beat changes in heart rate. The analysis of HRV, or heart rhythms, provides a powerful, noninvasive measure of neurocardiac function that reflects heart-brain interactions and autonomic nervous system dynamics, which are particularly sensitive to changes in emotional states. Research suggests that there is an important link between emotions and changes in the patterns of both efferent (descending) and afferent (ascending) autonomic activity. These changes in autonomic activity are associated with dramatic changes in the pattern of the heart's rhythm that often occur without any change in the amount of heart rate variability. Specifically, during the experience of negative emotions such as anger, frustration or anxiety, heart rhythms become more erratic and disordered, indicating less synchronization in the reciprocal action that ensues between the parasympathetic and sympathetic branches of the autonomic nervous system (ANS). In short-term (e.g., 3 to 10 seconds) responses to an unpleasant emotional experience, a heart rate deceleration will typically occur in the heart rhythm. In contrast, sustained positive emotions, such as appreciation, love or compassion, are associated with highly ordered or coherent patterns in the heart rhythms, reflecting greater synchronization between the two branches of the ANS, and a shift in autonomic balance toward increased parasympathetic activity. In short-term responses, a pleasant emotional experience may lead to an acceleration in the heart rate.

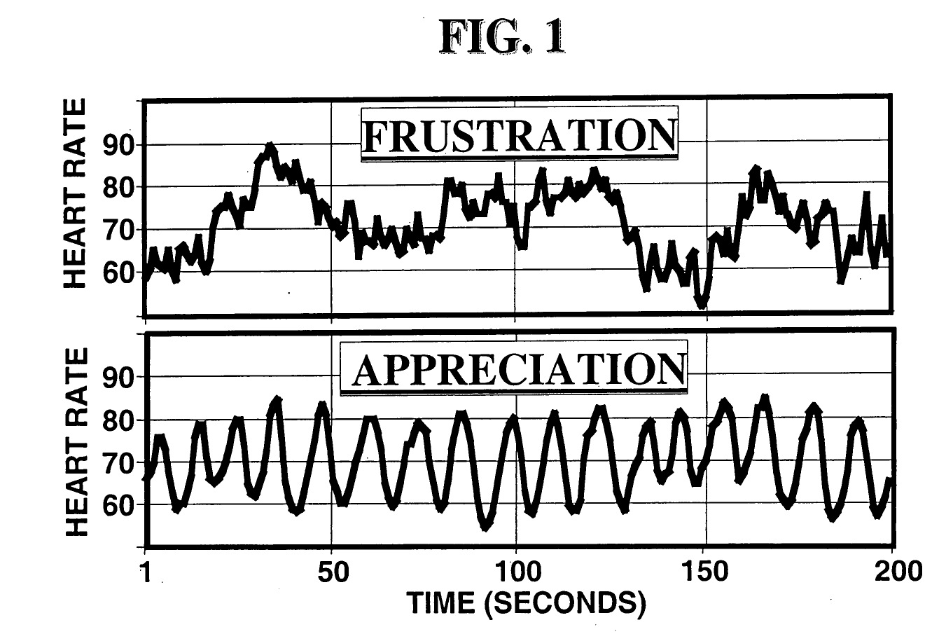

Referring to FIG. 1, heart rate variability (heart rhythm) patterns of an individual are depicted for both states of frustration and appreciation. In one embodiment, the state of appreciation may be achieved using a positive emotion refocusing exercise, such the Freeze-Frame technique previously mentioned and disclosed in U.S. Pat. No. 6,358,201, entitled “Method and Apparatus for Facilitating Physiological Coherence and Autonomic Balance,” issued on Mar. 19, 2002, and which is hereby incorporated by reference.

It is of note that when the recording is analyzed statistically, the amount of heart rate variability is found to remain virtually the same during the two different emotional states; however, the pattern of the heart rhythm changes distinctly. Note the erratic, disordered heart rhythm pattern associated with frustration versus the smooth, harmonious, sine-wave-like (coherent) pattern of an individual experiencing a heartfelt feeling of appreciation. This pattern is referred to as physiological coherence and is associated with a number of physiological and psychological benefits, including increased intuition.

The term “physiological coherence” may be used herein to describe a number of related physiological phenomena associated with more ordered and harmonious interactions among the body's systems and improved flow of information throughout the psychophysiological networks. The term coherence has several related definitions. A common definition of the term is “the quality of being logically integrated, consistent, and intelligible,” as in a coherent argument. In this context, thoughts and emotional states can be considered “coherent” or “incoherent.” Importantly, however, these associations are not merely metaphorical, as different emotions are in fact associated with different degrees of coherence in the oscillatory rhythms generated by the body's various systems.

The term “coherence” is used in physics to describe the ordered or constructive distribution of power within a waveform. The more stable the frequency and shape of the waveform, the higher the coherence. An example of a coherent wave is the sine wave. The term autocoherence is used to denote this kind of coherence. In physiological systems, this type of coherence describes the degree of order and stability in the rhythmic activity generated by a single oscillatory system. One embodiment for computing coherence is disclosed in previously-incorporated U.S. Pat. No. 6,358,201.

Coherence also describes two or more waves that are either phase- or frequency-locked. In physiology, coherence may be used to describe a functional mode in which two or more of the body's oscillatory systems, such as respiration and heart rhythms, become entrained and oscillate at the same frequency. The term cross-coherence may be used to specify this type of coherence.

Any one of the above definitions may be applied to the study of both emotional physiology and bioelectromagnetism. Entrainment may be observed between heart rhythms, respiratory rhythms, and blood pressure oscillations.

Another related phenomenon associated with physiological coherence is resonance. In physics, resonance may be used to refer to a phenomenon whereby an unusually large vibration is produced in a system in response to a stimulus whose frequency is identical or nearly identical to the natural vibratory frequency of the system. The frequency of the vibration produced in such a state is said to be the resonant frequency of the system. When the human system is operating in the coherent mode, increased synchronization occurs between the sympathetic and parasympathetic branches of the ANS, and entrainment between the heart rhythms, respiration and blood pressure oscillations may be observed. This occurs because these oscillatory subsystems are all vibrating at the resonant frequency of the system. Most models show that the resonant frequency of the human cardiovascular system is determined by the feedback loops between the heart and brain. In humans and in many animals, the resonant frequency is approximately 0.1 hertz, which is equivalent to a 10-second rhythm.

In short, the term coherence will be used as an umbrella term to describe a physiological mode that encompasses entrainment, resonance, and synchronization—distinct but related phenomena, all of which emerge from the harmonious activity and interactions of the body's subsystems. Correlates of physiological coherence include: increased synchronization between the two branches of the ANS, a shift in autonomic balance toward increased parasympathetic activity, increased heart-brain synchronization, increased vascular resonance, and entrainment between diverse physiological oscillatory systems. The coherent mode is reflected by a smooth, sine wave-like pattern in the heart rhythms (heart rhythm coherence) and a narrow-band, high-amplitude peak in the low frequency range of the heart rate variability power spectrum, at a frequency of about 0.1 hertz.

By applying spectral analysis techniques to the HRV waveform, its different frequency components, which represent the activity of the sympathetic or parasympathetic branches of the autonomic nervous system, can be discerned. The HRV power spectrum is divided into three frequency ranges or bands: very low frequency (VLF), 0.033 to 0.04 Hz; low frequency (LF), 0.04 to 0.15 Hz; and high frequency (HF), 0.15 to 0.4 Hz.

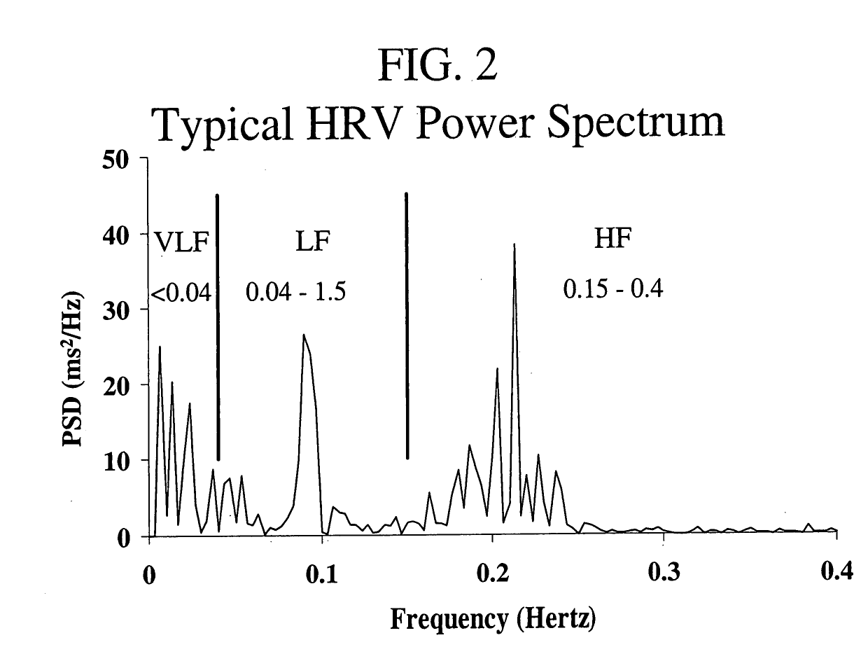

Referring now to FIG. 2, a typical HRV power spectrum is shown in which the typical VLF, LF and HF regions are denoted. The high frequency (HF) band is widely accepted as a measure of parasympathetic or vagal activity. The peak in this band corresponds to the heart rate variations related to the respiratory cycle, commonly referred to as respiratory sinus arrhythmia (RSA). Reduced parasympathetic activity has been found in individuals under mental or emotional stress, suffering from panic, anxiety or worry, depression, heart disease and many other disorders. As such, previous RSA training approaches have focused on increasing the HF peak in the HRV power spectrum. The low frequency (LF) region can reflect both sympathetic and parasympathetic activity, especially in short-term recordings.

II. Electrophysiological Intuition Indicator

It is commonly assumed among neuroscientists that mental concepts, conscious awareness, memory, and unconscious perception are emergent properties of the brain and nervous system. Thus, it is assumed that the mind is essentially a complex, dynamical system subject to the same physical constraints as is all matter.

Within physics, however, an absolute direction of time is far less certain (e.g., general relativity, electrodynamics and quantum mechanics). These non-local effects are generally assumed to manifest only in subatomic realms. However, macroscopic scale examples have been reported throughout history (e.g., prophesy, precognition, intuition).

Of particular interest is the intuitive hunch, commonly described as a “bad feeling” with no evident cause, occurring before an unexpected emotional event. It has been determined that if a future event is sufficiently important, novel, or emotional, it may precipitate a change in the present physiological state that is consistent with the future reaction.

To that end, one aspect of the invention is to detect and quantify the ability of an individual to experience an electrophysiological response to a future or unknown event that is consistent with the actual outcome. Another aspect of the invention is to quantify the electrophysiological responses for a group of individuals as a predictor of future events and/or to answer an unknown question.

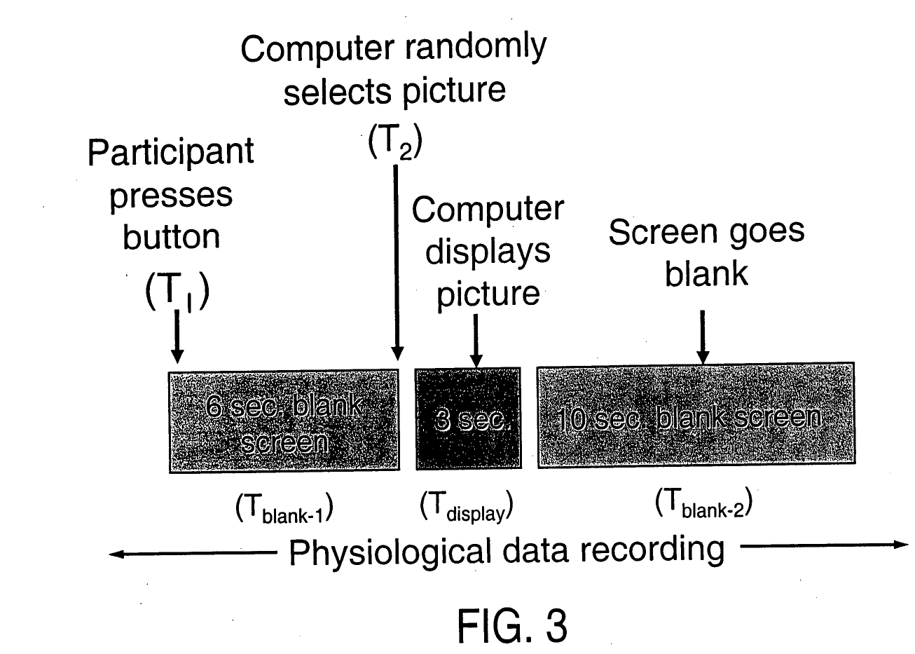

Referring now to FIG. 3, one embodiment of the procedure for implementing an intuition indicator is depicted. In this embodiment, a participant is connected to a system which monitors one or more electrophysiological properties (e.g., HRV, EEG, respiration pattern, SCL, etc.). In one embodiment, the EEG properties of a participant may be measured by fitting each participant with EEG electrodes applied to the sites as defined by the International 10-20 System. For measuring skin conductance, surface silver-silver chloride electrodes may be attached to the participant's hand and/or fingers. Respiration may be measured using a respiration belt placed around the participant's chest. And finally, HRV may be derived from the ECG or pulse wave (but not limited to). It should be appreciated that an ECG amplifier may be used, and that a photoplethysmographic sensor may also be attached to the participant to measure pulse transit time in order to determine changes in blood pressure and to determine the time at which the blood pressure wave reaches the brain.

The procedure begins with the individual pressing an activation button at point T₁. A pretermined period of time (T_{blank-1}) then passes before the system randomly selects a stimulus (e.g., image, a sound, question, etc.) for display at T₂. While in the embodiment of FIG. 3 T_{blank-1} is 6 seconds, it should of course be appreciated that T_{blank-1} may be any length of time. In another embodiment, T_{blank-1} is also randomly selected.

Continuing to refer to FIG. 3, in this embodiment the system provides the randomly selected stimulus for 3 seconds (T_{display}), although any other length of time may similarly be selected. After T_{display}, the stimulus is removed for an additional predetermined period of time (T_{blank-2}). While T_{blank-2} is 10 seconds in the embodiment of FIG. 3, any other length of time may be used. As mentioned above, the electrophysiological data for multiple individuals may be simultaneously monitored during the above-described procedure. In such a case, a combined value of the groups electrophysiological data may be determined and used in a predictive model.

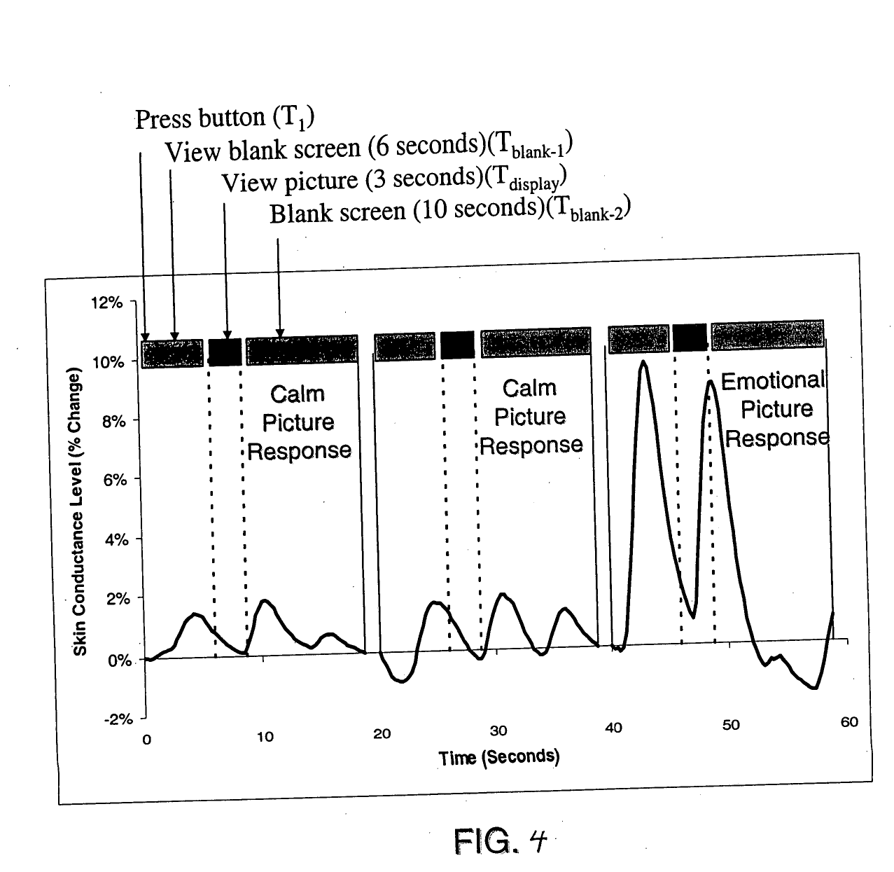

FIG. 4 depicts sample data produced from the procedure of FIG. 3. In this embodiment, physiological data that was recorded during the T_{blank-1}, T_{display} and T_{blank-2} time periods was plotted versus time. In particular, FIG. 4 depicts a graph of time plotted versus the percentage change in SCL. As with FIG. 3, the subject (or group of subjects) presses an initiation button at T₁, views a blank screen for T_{blank-1}, is exposed to the stimulus for T_{display}, and then views a blank screen again for T_{blank-2}. However, unlike FIG. 3, FIG. 4 includes response data for three separate stimuli, where the first two are low-level stimuli (e.g., calm pictures) and the third is a high-level stimuli (e.g., emotional picture).

In one embodiment, independent component analysis (ICA) was used to remove eye blinks from the raw EEG data. Randomized paired sample permutation t test multivariate analysis may also be used to test for significant differences between calm and emotional trials.

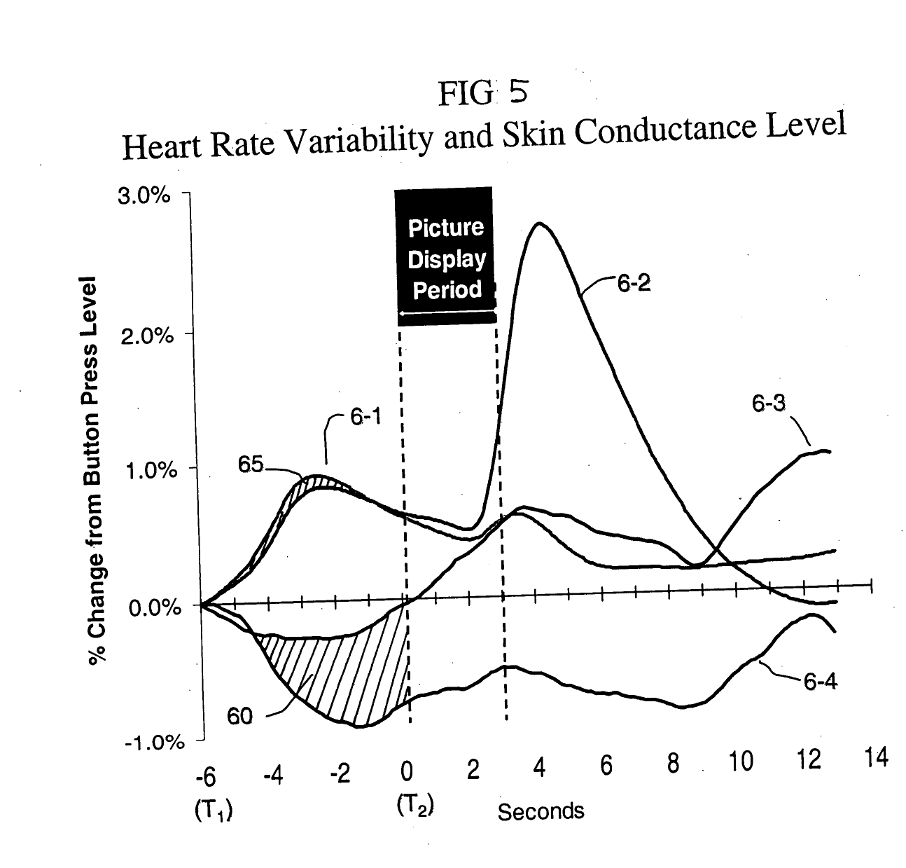

In yet another illustration of measurements of electrophysiological data, FIG. 5 shows a graph of time versus the percent change in both HRV and SCL. In particular, plot 6-1 is the SCL response curve for the low-level stimuli, whereas plot 6-2 is the SCL response curve (heart rate deceleration) for the high-level stimuli. In addition, plot 6-3 is the HRV response curve for the low-level stimuli, whereas plot 6-4 is the HRV response curve (heart rate deceleration) for the high-level stimuli.

Area 60 represents a measurement of intuition as measured by the percentage change in a subject's HRV from the time an initiation button is pressed (T₁) to the time the stimulus is provided (T₂). In contrast, area 65 represents one way to measure a subject's ability to “sense” a future event based on the percentage change in the subject's SCL leading up to the event in question. In sum, the data of FIG. 5 suggests that HRV provides a more pronounced electrophysiological measurement of intuition than does SCL.

The technique referred to herein as “signal averaging” may be used for detecting response patterns in biological systems and providing an electrophysiological background measurement to which current nervous system response can be compared. In this manor a measure of intuition can be obtained. In essence, signal averaging is a digital technique for separating a repetitive signal from noise without introducing appreciable signal distortion. In one embodiment, signal averaging is accomplished by superimposing any number of equal-length epochs, each of which contains a repeating periodic signal. This procedure emphasizes and distinguishes any signal that is time-locked to the periodic signal, while also eliminating variations that are not time-locked. In the embodiment where signal averaging is used to detect EEG activity that is time-locked to the ECG, the resultant waveform shall be referred to as the “heartbeat evoked potential.”

In one embodiment signal averaging may be performed by first digitizing the signals recorded from the EEG and ECG. Thereafter, the R-wave (peak) of the ECG may be used as the time reference for cutting the EEG and ECG signals into individual segments. In one embodiment, these individual segments may then be averaged together to produce the resultant heartbeat evoked potential waveforms. In the multi-subject embodiment, the above signal averaging procedure may be carried out for the group and the resulting waveforms used as the predictive measure.

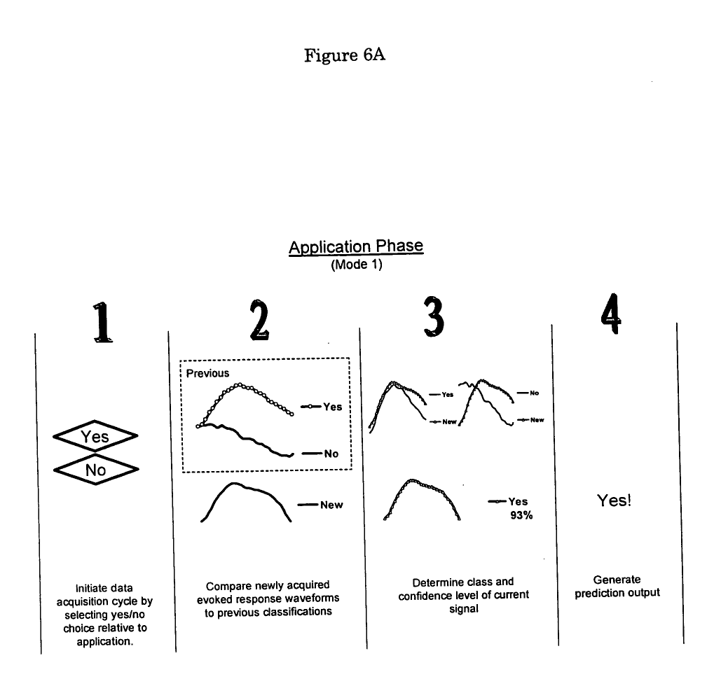

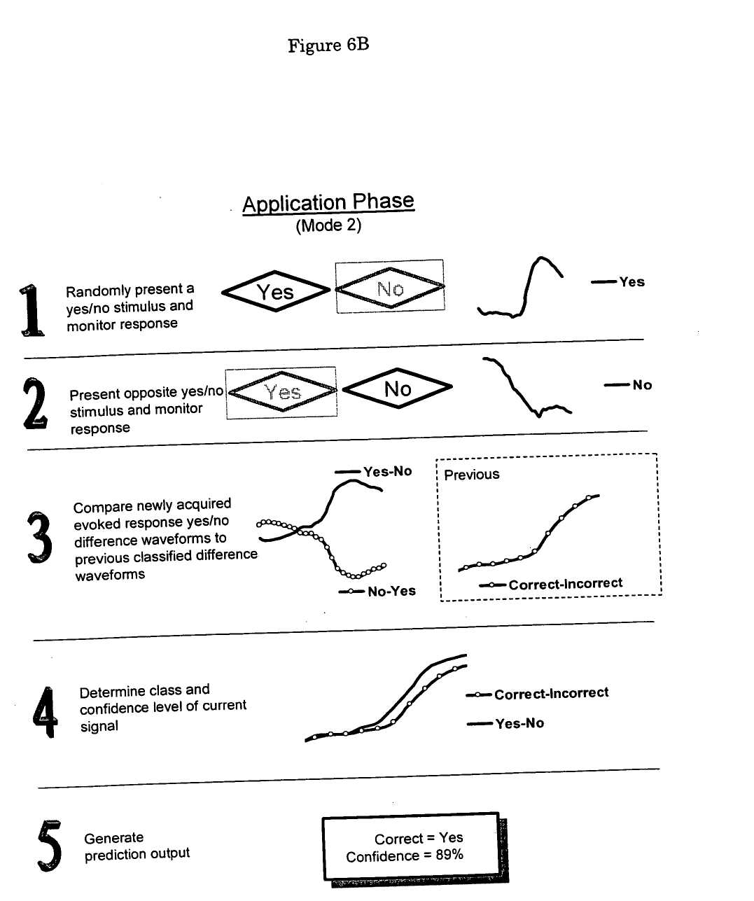

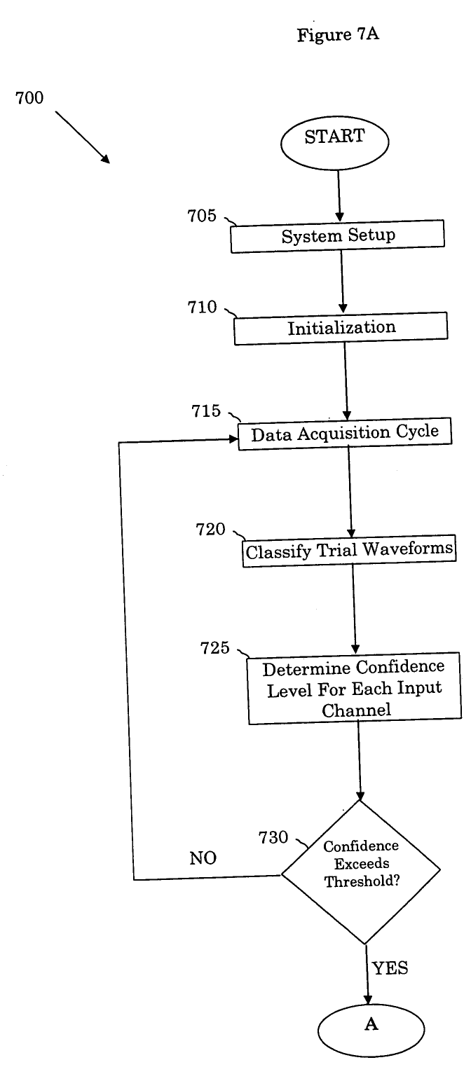

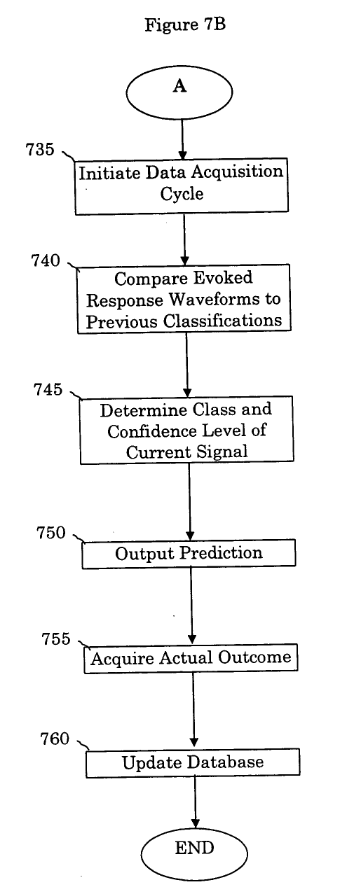

FIGS. 6A-6B depict two embodiments of operational modes consistent with the principles of the invention. In the embodiment of FIG. 6A, referred to hereinafter as Mode 1, a subject may choose an answer or guess at what the future outcome will be or the answer to an unknown question as the first phase of the process (Phase 1). The physiological data from all the sensors may then be analyzed following this stimulus (in this embodiment the choice is the stimulus) to see which measures and/or combination of measures best predicts the actual outcome (discuss in detail below with reference to FIGS. 7A-7B). While in the embodiment of FIG. 6A a yes/no construct is used, it should of course be understood that any form of opposing questions may similarly be used (e.g., red/black, up/down, heads/tails, buy/sell, sick/healthy, etc.).

Continuing to refer to FIG. 6A, and as will be discussed in more detail below with reference to FIGS. 7A-7B, phase 2 of Mode 1 involves comparing the newly acquired evoked response waveforms to previous classification. Phase 3 involves determining class and confidence levels of the current signal, and phase 4 involves generation of the predictive output.

FIG. 6B depicts a second embodiment of an operational mode (Mode 2). With Mode 2, the individual is separately presented with a ‘Yes’ and ‘No’ indicator in random order. The physiological data from all the sensors may then be analyzed following the presentation of the stimulus to see which measures and combination of measures best predicts the actual outcome. In Mode 2, the presentation of the stimulus acts as the initiation of data cycle (although data is being recorded prior to the stimulus). In addition, both this pre-stimulus data as well as the post-stimulus data may also be used in the analysis.

As shown in FIG. 6B, phase 1 of Mode 2 involves the random presentation of a yes/no stimulus. Then, at phase 2, the opposite stimulus may be presented. Thereafter, in the embodiment of FIG. 6B, the newly acquired evoked response waveforms may be compared to previous classifications at phase 3, while the class and confidence level of the current signal may be determined at phase 4. Finally, the predictive output may be generated at phase 5.

It should be appreciated that either Mode 1 or Mode 2 may be calibrated to either an randomly generated internal outcome source (e.g., internal random number generator) or an actual outcome generated by an event occurring in the outside environment (e.g., flipping a coin, stock price changes, etc.). It should further be appreciated that the time intervals between the various phases of the selected operational mode may be user-determined.

FIG. 7A is a flow diagram for one embodiment of a calibration phase for a system of carrying one or more aspects of the invention. In particular, process 700 begins with the system's setup at block 705. Once the appropriate electrodes and sensors are connected to a subject, in one embodiment the system may check to insure that the various signals are being adequately acquired and that the quality of the signals are adequate for analysis. By way of a non-limiting example, the resistance values of the EEG, ECG and skin conductance electrodes may be checked to insure they are low enough. In addition, the signals produced by such electrodes may similarly be checked to verify that the signals are at expected levels. In one embodiment, if one or more of the signals are not being adequately acquired, the system may alert the user. In another embodiment, or in addition to one or more of the previous embodiments, once all the signal levels are confirmed, the system may auto calibrate and normalize the various signals in preparation for data acquisition.

Process 700 continues to the initialization operation of block 710. In one embodiment, previous values and confidence levels may be reset in preparation for the new calibration. In one embodiment, part of the initialization process involves selecting an operational mode prior to data acquisition and calibration to the individual person and context of the predictions to be made. While it should be appreciated that there are numerous operational modes envisioned, FIGS. 6A-6B above illustrated such two exemplary operational modes.

At block 715 of FIG. 7A, the process 700 continues with the data acquisition. If the system is set to Mode 1 (see FIG. 7A above), the moment the Yes/No choice is made (e.g., subject presses button), the cycle may be initiated. In Mode 2, however, the cycle may be initiated when the choice is randomly presented to the subject (e.g., phase 1 and 2). The data collected from all the sensors may then be stored in memory. In one embodiment, the outcome may then be determined (either through an internal random number generator or the outcome from an external source) and also stored in memory. In another embodiment, the data from each sensor is then appropriately processed and compared to previously collected data relating to a known outcome.

It should be noted that examples of the physiological signals that can be analyzed include changes in skin conductance, EEG derivatives (which are evoked potentials where the slope and degree on negativity and onset of the positive shift occur), and heartbeat evoked potentials. Moreover, the derivatives from the ECG or pulse sensors are heart rate accelerations and/or decelerations that may similarly be examined. It should be appreciated that numerous other physiological measures may similarly be examined (e.g., pulse amplitude, blood pressure, etc.).

Continuing to refer to FIG. 7A, process 700 continues to block 720 where the trial waveforms may be classified according to the predicted and actual outcomes. In one embodiment, the waveforms from the current cycle may be compared to the averaged waveforms obtained in previous cycles (e.g., Mode 1-phase 2 and Mode 2-phase 3). At block 725, the confidence level of the predicted outcome may be determined by comparing each of the signals and their derivatives to the data collected in previous cycles and the actual outcomes. In addition, the current level of physiological coherence could also influence the confidence level. In one embodiment, the combination of measures which has the most predictive power in previous trials may also be determined and compared to the current cycle and used in the determination of the confidence level output.

At this point, process 700 continues to decision block 730 where a determination may be made as to whether or not the confidence level exceeds a predetermined threshold. If not, process 700 initiates an additional calibration cycle and the process described above (blocks 715-725) is repeated until sufficient data has been obtained that the confidence level exceeds the current minimum threshold setting. If, on the other hand, the minimum threshold is reached, then process 700 continues to the application phase of FIG. 7B. In one embodiment, the user may be provided with a notification that the calibration phase is complete and that the application phase will be commenced.

Referring now to FIG. 7B, the application phase of process 700 begins with block 735 and the initiation of the data acquisition cycle. Depending on the mode selected (e.g., Mode 1, Mode 2, etc.), the system or the subject may provide the stimulus that initiates the application cycle. Once the data acquisition cycle has been initiated, the evoked response waveforms may be compared to previous classifications at block 740. In one embodiment, the waveforms and their derivatives may be compared to the average waveforms built up and stored during the calibration phase.

In the embodiment of FIG. 7B, process 700 continues with block 745 where the type of signals and the confidence level of the current cycle may be determined. The prediction may then be generated and output to a user interface (block 750), which may be a computer screen, an indicator light, a tactile indicator, etc. Moreover, the actual outcome, once determined, may optionally be inputted into the system (block 755). The database may then be updated with the actual outcome and the physiological data (block 760). It should be appreciated that predictive outcomes can be improved by selecting those subjects which exhibit a superior ability to generate good predictive outcomes based on there physiological data.

It should further be appreciated that, while some of the above discussion was in terns of human subjects, the principles of the invention may similarly be applied to animals as well. For example, there has been data to suggest that dogs can predict the onset of seizers in there owners, or the moment their owners decided to come home. Similarly, the principles of the invention may similarly be applied on a cellular level.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

1. A method for detection and measurement of intuition comprising:
measuring an electrophysiological property of a subject at a first point in time;
measuring said electrophysiological property of said subject at a second point in time;
calculating a measure of change of said electrophysiological property between said first point in time and said second point in time; and,
determining an event to occur at a third point in time based on said measure.

2. The method of claim 1 wherein said measuring said electrophysiological property comprises measuring said electrophysiological property of the subject at the first point in time, and measuring the electrophysiological property at the second point in time, where said electrophysiological property is at least one of heart rate variability, brain wave activity, skin conductance level and respiration pattern.

3. The method of claim 2 wherein said measuring said electrophysiological property comprises measuring said subject's heart rate variability at a first point in time, and measuring said subject's heart rate variability at a second point in time, said heart rate variability to be derived from an electrocardiogram or pulse signal and to be a measure of the beat-to-beat changes in the subject's heart rate.

4. The method of claim 1 wherein the difference between said first point in time and said second point in time is between 3 seconds and 10 seconds.

5. The method of claim 1 wherein said calculating comprises calculating a measure of change of said electrophysiological property between said first point in time and said second point in time, said measure to be based on the percentage change of said electrophysiological property between said first and second points in time.

6. The method of claim 1 further comprising:
monitoring said electrophysiological property for a period of time;
plotting changes in said electrophysiological property as a function of time;
interpreting said plotting to determine said event.

7. The method of claim 1 wherein said measuring comprises measuring a collective electrophysiological property for a plurality of subjects at the first point in time, and measuring said collective electrophysiological property for said plurality of subjects at the second point in time, said collective electrophysiological property to be based on electrophysiological properties for each of said plurality of subjects.

8. The method of claim 7 wherein said calculating the measure comprises calculating the measure of change of said collective electrophysiological property for said plurality of subjects between said first point in time and said second point in time.

9. The method of claim 1 further comprising exposing said subject, prior to said measuring, to a stimulus associated with said event.

10. The method of claim 9 wherein said exposing comprises exposing said subject, prior to said measuring, to a visual stimulus representative of said event.

11. A system for detection and measurement of intuition comprising:
a human subject;
a means for measuring an electrophysiological property of said human subject at a first point in time;
a means for measuring said electrophysiological property of said human subject at a second point in time;
a means for calculating a measure of change of said electrophysiological property between said first point in time and said second point in time; and,
a means for predicting an event to occur at a third point in time based on said measure.

12. The system of claim 11 wherein said electrophysiological property is at least one of heart rate variability, brain wave activity, skin conductance level and respiration pattern.

13. The system of claim 12 wherein said electrophysiological property is said subject's heart rate variability, said heart rate variability to be derived from an electrocardiogram or pulse signal and to be a measure of the beat-to-beat changes in the subject's heart rate.

14. The system of claim 11 wherein said measure is based on the percentage change of said electrophysiological property between said first and second points in time.

15. The system of claim 11 wherein said means for measuring comprises means for measuring a collective electrophysiological property for a plurality of human subjects at the first point in time, and measuring said collective electrophysiological property for said plurality of human subjects at the second point in time, said collective electrophysiological property to be based on electrophysiological properties for each of said plurality of human subjects.

16. The system of claim 15 wherein said means for calculating the measure comprises means for calculating the measure of change of said collective electrophysiological property for said plurality of human subjects between said first point in time and said second point in time.

17. The system of claim 11 further comprising means for exposing said human subject, prior to said measuring, to a stimulus associated with said event.

18. A method for detection and measurement of intuition comprising:
exposing a subject to a stimulus associated with one of a future event;
monitoring a electrophysiological property of said subject over a period of time, said period of time to precede said future event;
calculating a measure of change of said electrophysiological property over said period of time; and,
determining an attribute of said future event based on said measure of change.

19. The method of claim 18 wherein said monitoring the electrophysiological property comprises monitoring the electrophysiological property of said subject over said period of time, where said electrophysiological property is at least one of heart rate variability, brain wave activity, skin conductance level and respiration pattern.

20. The method of claim 18 wherein said calculating comprises calculating the measure of change of said electrophysiological property over the period of time, said measure to be based on the percentage change of said electrophysiological property over said period of time.

21. The method of claim 18 wherein said monitoring comprises monitoring a collective electrophysiological property for a plurality of subjects over said period of time, said collective electrophysiological property to be based on electrophysiological properties for each of said plurality of subjects.

22. The method of claim 21 wherein said calculating the measure of change comprises calculating the measure of change of said collective electrophysiological property for said plurality of subjects over said period of time.

23. A method comprising:
sampling a physiological characteristic of a subject;
determining a measure of said physiological characteristic; and
comparing said measure to a physiological coherency range to determine if said subject is in a state of physiological coherency, said state being characterized by a sine-wave-shaped heart rhythm pattern and an increased synchronization between two or more oscillatory systems of said subject.

24. The method of claim 23 wherein said determining comprises determining the measure of said physiological characteristic where said physiological characteristic comprises heart rate variability, respiration patterns, blood pressure rhythms and ECG-R wave amplitudes.

25. The method of claim 23 wherein said determining comprises determining the measure of said physiological characteristic, said measure being expressed in one of a frequency domain, a time domain, a period analysis and a template match.

26. The method of claim 23 wherein said oscillatory systems are selected from the group consisting of heart rhythms, respiratory rhythms and blood pressure oscillations, ECG R-wave amplitude, pulse wave, impedance measures and vascular responses.

27. The method of claim 23 wherein said state of physiological coherency is further characterized by a state of entrainment between said subject's heart rhythms and respiration rhythms.

28. The method of claim 23 wherein said state of physiological coherency is further characterized by increased positive emotions in said subject.

29. The method of claim 23 wherein said coherency range is expressed in the frequency range and is between 0.03125 Hertz 0.234 Hertz.

30. The method of claim 29, wherein said coherency range includes a resonance frequency of said physiological characteristic.

31. The method of claim 23 wherein said measure is a pattern usable to determine an emotional state of said subject.

32. The method of claim 23 wherein said state of physiological coherency comprises having one of a phase and frequency lock between said two or more oscillatory systems of said subject.

33. The method of claim 23 wherein, after said comparing the measure to the physiological coherency range, the method further comprises providing said subject with feedback based on said comparing.

34. The method of claim 33 wherein said feedback causes said subject to enter said state of physiological coherency.

35. The method of claim 23 further comprising:
sampling the physiological characteristics from each of a plurality of subjects;
determining a group measure from said sampling of the physiological characteristic from each of said plurality of subjects; and
comparing said group measure to the physiological coherency range to determine if said plurality of subjects are in the state of physiological coherency.

36. The method of claim 35 wherein, after said comparing the group measure to the physiological coherency range, the method further comprises providing said plurality of subjects with feedback based on said comparing.

37. The method of claim 36 wherein said feedback causes said plurality of subjects to move closer to said state of physiological coherency.

38. A system comprising:
sampling means adapted to sample a physiological characteristic of a subject; and,
a processor coupled to the sampling means, said processor to,
determine a measure of said physiological characteristic, and
compare said measure to a physiological coherency range to determine if said subject is in a state of physiological coherency, said state being characterized by a sine-wave-shaped heart rhythm pattern and an increased synchronization between two or more oscillatory systems of said subject.

39. The system of claim 38 wherein said physiological characteristic comprises heart rate variability, respiration patterns, blood pressure rhythms and ECG-R wave amplitudes.

40. The system of claim 38 wherein said measure is expressed in one of a frequency domain, a time domain, a period analysis and a template match.

41. The system of claim 38 wherein said oscillatory systems are selected from the group consisting of heart rhythms, respiratory rhythms and blood pressure oscillations, ECG R-wave amplitude—pulse wave, impedance measures, vascular responses.

42. The system of claim 38 wherein said state of physiological coherency is further characterized by a state of entrainment between said subject's heart rhythms and respiration rhythms.

43. The system of claim 38 wherein said state of physiological coherency is further characterized by increased positive emotions in said subject.

44. The system of claim 38 wherein said coherency range is expressed in the frequency range and is between 0.03125 Hertz 0.234 Hertz.

45. The system of claim 44, wherein said coherency range includes a resonance frequency of said physiological characteristic.

46. The system of claim 38 wherein said measure is a pattern usable to determine an emotional state of said subject.

47. The system of claim 38 wherein said state of physiological coherency comprises having one of a phase and frequency lock between said two or more oscillatory systems of said subject.

48. The system of claim 38 wherein the processor is further to provide said subject with feedback based on a result of comparing said measure to the physiological coherency range.

49. The system of claim 48 wherein said feedback causes said subject to enter said state of physiological coherency.

50. The system of claim 38 wherein the sampling means is further adapted to sample the physiological characteristic from each of a plurality of subjects, and wherein the processor is further to,
determine a group measure from said sampling of the physiological characteristic from each of said plurality of subjects, and
compare said group measure to the physiological coherency range to determine if said plurality of subjects are in the state of physiological coherency.

51. The system of claim 50 wherein, after said processor compares the group measure to the physiological coherency range, the processor is further to provide said plurality of subjects with feedback based on a result of comparing the group measure to the physiological coherency range.

52. The system of claim 51 wherein said feedback causes said plurality of subjects to move closer to said state of physiological coherency.
